(19) United States
(12) Patent Application Publication
(10) Pub. No.: US 2024/0266027 A1
(43) Pub. Date: Aug. 8, 2024

(54) MEDICAL INFORMATION PROCESSING DEVICE, MEDICAL INFORMATION PROCESSING SYSTEM, AND NON-TRANSITORY COMPUTER READABLE MEDIUM

(71) Applicant: CANON MEDICAL SYSTEMS CORPORATION, Otawara-shi (JP)

(72) Inventors: Koichi TERAI, Shioya (JP); Masahiro YOSHIWAKI, Otawara (JP); Taro TAKOSHIMA, Nasushiobara (JP); Ichirou MAEDA, Otawara (JP); Hiroyuki YAMASAKI, Yaita (JP); Shuhei BANNAE, Utsunomiya (JP)

(73) Assignee: CANON MEDICAL SYSTEMS CORPORATION, Otawara-shi (JP)

(21) Appl. No.: 18/434,119

(22) Filed: Feb. 6, 2024

(30) Foreign Application Priority Data
Feb. 6, 2023 (JP) 2023-016332

(51) Int. Cl. G16H 30/20

(52) U.S. Cl. CPC G16H 30/20

(57) ABSTRACT

A medical information processing device includes a memory and a processor. The processor is configured to receive a medical image; store, in the memory, information on the medical image and information on processing executed on the medical image in association with each other; determine processing to be executed based on the information on the medical image and the information on the processing stored in the memory; and execute, upon receiving the medical image, the processing to be executed determined based on the information on the medical image.

### CROSS REFERENCE TO THE PRIOR APPLICATIONS

This application is based upon and claims the benefit of priority from the Japanese Patent Application No. 2023-016332, filed on Feb. 6, 2023, the entire contents of which are incorporated herein by reference.

### FIELD

Embodiments disclosed in the present specification and the drawings relate to a medical information processing device, a medical information processing system, and a program.

### BACKGROUND

The processing of a medical image is one of determination criteria in subsequent diagnosis and medical treatment, and appropriate and high-speed processing of image data output from an imaging device is required. For example, a medical information processing device uses reception of the medical image as a trigger, specifies an image analysis application to be executed according to a preset rule, and automatically executes the application.

In such a medical information processing device, regarding the rule, it is very complicated to perform settings that define which series of imaging protocols of which imaging device an application is to run on, and a large number of man-hours are required for installation and maintenance of the device.

### BRIEF DESCRIPTION OF DRAWINGS

FIG. 1 is a block diagram schematically illustrating an example of a medical information processing device according to an embodiment;

FIG. 2 is a flowchart illustrating an example of processing of the medical information processing device according to the embodiment;

FIG. 3 is a flowchart illustrating an example of the processing of the medical information processing device according to the embodiment;

FIG. 4 is a flowchart illustrating an example of the processing of the medical information processing device according to the embodiment;

FIG. 5 is a flowchart illustrating an example of the processing of the medical information processing device according to the embodiment;

FIG. 6 is a flowchart illustrating an example of the processing of the medical information processing device according to the embodiment; and

FIG. 7 is a block diagram schematically illustrating an example of a medical information processing system according to the embodiment.

### DETAILED DESCRIPTION

According to one embodiment, a medical information processing device includes a memory and a processor. The processor is configured to receive a medical image; store, in the memory, information on the medical image and information on processing executed on the medical image in association with each other; determine processing to be executed based on the information on the medical image and the information on the processing stored in the memory; and execute, upon receiving the medical image, the processing to be executed determined based on the information on the medical image.

Hereinafter, embodiments will be described with reference to the drawings. In the present disclosure, in a mode in which information processing by software is specifically implemented by using hardware resources, the software is executed by a program, and the software can be implemented by the program itself or a non-transitory computer readable medium storing the program. Furthermore, in the various data in the present specification and the drawings, data mainly used by an information processing device is typically digital data.

The medical information processing device according to the present disclosure transitions to a step of determining appropriate processing to be automatically executed for a medical image by transitioning to several steps of selecting the processing to be automatically executed for the medical image. In the present disclosure, the medical image is not limited to one image, and may be time-series data including a plurality of frames, or may be a plurality of pieces of image data suitable for some processing. In addition, the medical image may include data that can be appropriately referred to as a medical image.

This step can include, for example, a step in which a user inputs processing to be applied to the medical image to the medical information processing device, a step in which the medical information processing device proposes the processing to be applied to the medical image to a user, and a step in which the medical information processing device automatically determines and executes the processing to be applied to the medical image.

In the following description, unless otherwise specified, a mode including the above-described three steps will be described as an embodiment, but these steps are not all essential, and for example, one of the step of inputting processing by the user and the step of proposing processing to the user can be omitted.

### First Embodiment

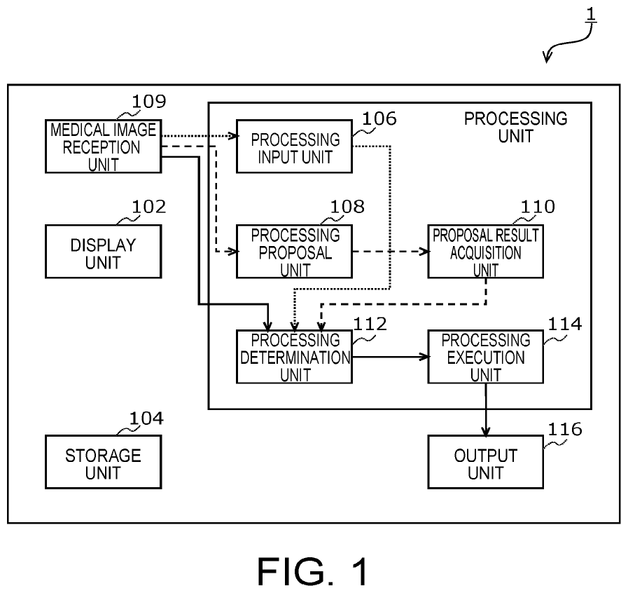

FIG. 1 is a block diagram schematically illustrating an example of a medical information processing device according to an embodiment. A medical information processing device 1 includes a medical image reception unit 100, a display unit 102, a storage unit 104, a processing input unit 106, a processing proposal unit 108, a proposal result acquisition unit 110, a processing determination unit 112, a processing execution unit 114, and an output unit 116. In addition, the medical information processing device 1 includes a control unit that controls an overall configuration, a power supply unit that supplies power, and the like, which are not illustrated, as necessary.

The medical image reception unit 100 operates as a medical image reception unit in the medical information processing device 1, and receives a medical image from a sensor or the like provided outside. The medical image reception unit 100 can receive a medical image not from the sensor or the like but from another medical information processing device. The medical image may be a series of images. This medical image is, for example, an image including metadata conforming to the digital imaging and communications in medicine (DICOM) standard.

The metadata as an example of the information on the medical image is, for example, data including information (type information or the like) related to an imaging device, information related to an imaging method, and information such as an imaging parameter. Furthermore, the metadata may include, more specifically, data such as an identifier allocated to each imaging device and information related to each individual patient. By using such metadata, it is possible to determine processing in a general range such as imaging devices having the same type and the imaging device, and it is also possible to determine processing in a more specific range such as an individual difference between individual devices in the imaging devices having the same type and a difference depending on an individual patient.

The display unit 102 operates as a display unit in the medical information processing device 1, and includes a display or the like capable of displaying any information in the medical information processing device 1.

The storage unit 104 operates as a storage unit in the medical information processing device 1, and includes any storage device such as a memory and a storage, and a storage module capable of storing any information in the medical information processing device 1. The storage unit 104 is not limited to being provided inside the medical information processing device 1, and a part thereof may be provided outside the medical information processing device 1.

The processing input unit 106, the processing proposal unit 108, the proposal result acquisition unit 110, the processing determination unit 112, and the processing execution unit 114 may be integrated as one processing unit. At least a part of the processing unit may be formed by a general-purpose circuit, or at least a part thereof may be formed by a dedicated circuit. The processing execution unit 114 may be partially or entirely provided outside the medical information processing device 1.

The processing input unit 106 operates as a processing input unit in the medical information processing device 1, and receives processing to be executed on the received medical image input by a user. The display unit 102 can display, for example, information related to the received medical image, for example, metadata attached to the image, or the input image itself. On the other hand, the user can instruct processing to be executed on the medical image via the processing input unit 106, and the processing input unit 106 receives the processing input from the user.

The processing proposal unit 108 operates as a processing proposal unit in the medical information processing device 1 and proposes a processing candidate to be executed on the received medical image to the user. This proposal may be executed via the display unit 102.

The proposal result acquisition unit 110 operates as a proposal result acquisition unit in the medical information processing device 1, and acquires whether the user has selected the processing proposed by the processing proposal unit 108 and which processing the user has selected.

The processing determination unit 112 operates as a processing determination unit in the medical information processing device 1, and determines processing according to an output from the processing input unit 106, the proposal result acquisition unit 110, or the medical image reception unit 100 according to processing.

In first processing in which the user designates processing for the received medical image, the processing determination unit 112 determines processing designated by the user and received by the processing input unit 106 as processing to be executed on the received medical image.

In second processing of proposing processing to the user for the received medical image, the processing determination unit 112 determines processing selected by the user and acquired by the proposal result acquisition unit 110 as processing to be executed on the received medical image.

In third processing of automatically executing processing on the received medical image, the processing determination unit 112 determines processing to be automatically executed on the medical image from the metadata of the medical image received by the medical image reception unit 100.

In the processing unit of FIG. 1, a path indicated by a dotted line is a path for determination of processing in the first processing, a path indicated by a broken line is a path for determination of processing in the second processing, and a path indicated by a solid line is a path for determination of processing in the third processing.

The processing execution unit 114 operates as a processing execution function in the medical information processing device 1, and executes the processing determined by the processing determination unit 112 on the medical image. A part or all of the processing execution unit 114 may exist outside the medical information processing device 1. For example, in a case where processing that cannot be handled in the medical information processing device 1 and can be processed in another device is selected by the processing determination unit 112, the medical information processing device 1 can execute processing of the received medical image by transmitting the received medical image to the other device and requesting processing execution in the other device.

The output unit 116 operates as an output function in the medical information processing device 1, and can freely and selectively output the processed medical image processed by the processing execution unit 114 to the outside of the medical information processing device 1.

It is noted that, for example, the output unit 116 can not only output data to the outside of the medical information processing device 1 but also store processed data in the storage unit 104 of the medical information processing device 1. That is, the medical information processing device 1 may output the processed medical image to the outside immediately after performing processing, or may store the processed medical image in the storage unit 104 and allow the user to acquire the processed medical image at an appropriate timing.

The medical image reception unit 100 and the output unit 116 can include, for example, any interface such as a user interface such as an input unit or a display unit, a communication interface such as a communication unit, or the like.

Next, a flow of processing of the medical information processing device 1 will be described.

The medical information processing device 1 is a device that executes automatic processing according to a medical image and acquires a result of the execution. The medical information processing device 1 acquires metadata of the received medical image, and automatically executes processing when processing according to the metadata can be automatically acquired.

When the medical information processing device 1 cannot automatically execute the processing on the medical image based on a condition, the medical information processing device 1 receives a processing instruction from the user. The processing instruction from the user may be configured in two steps. When some processing candidates can be acquired based on the metadata, the medical information processing device 1 can present this candidate processing to the user. When the candidate processing cannot be acquired, the medical information processing device 1 receives an input of processing for the medical image from the user.

In the first processing which is an initial step, upon receiving the medical image, the medical information processing device 1 receives a processing instruction from the user, and stores, in the storage unit 104, the instructed processing and the metadata of the medical image in association with each other as user-designated data.

In the second processing which is the next step, upon receiving the medical image, the medical information processing device 1 proposes one or a plurality of types of processing candidates based on the metadata of the medical image to the user, and executes processing selected by the user. The medical information processing device 1 stores, in the storage unit 104, the processing selected by the user and the metadata of the medical image in association with each other as proposal result data.

In the third processing which is the final step, upon receiving the medical image, the medical information processing device 1 automatically executes processing based on the metadata of the medical image.

The first processing to the third processing are set according to the metadata of the medical image, and the medical information processing device 1 executes the processing according to the received medical data and the processing. As an example, the processing transitions from the first processing to the second processing and from the second processing to the third processing. As another example, the processing may directly transition from the first processing to the third processing.

Processing executed on the medical image by the medical information processing device 1 and transition of the processing will be described in detail using a flowchart.

The medical information processing device 1 first acquires a medical image to be processed via the medical image reception unit 100 (S100).

As described above, the medical image may be a series of images formed in a temporal direction and/or a spatial direction. Of course, the medical image may be formed from one image. The medical image may be an image acquired using any source such as an image acquired using infrared rays, an image acquired using ultraviolet rays, an image acquired using X-rays, an image acquired by tomography, an image acquired by ultrasound, or the like.

Upon receiving the medical image via the medical image reception unit 100, the medical information processing device 1 branches processing using metadata given to the medical image. A branching condition is a condition based on whether processing can be proposed for the medical image and whether processing can be automatically determined for the medical image.

The medical information processing device 1 refers to the data stored in the storage unit 104 to determine whether the received medical image satisfies a first condition (S102). The first condition is a condition for determining a state in which processing to be executed on the received medical image can be proposed or the processing to be executed thereon can be automatically determined. That is, the medical information processing device 1 determines that the first condition is not satisfied (S102: NO) when the processing to be executed on the received medical image cannot be proposed and cannot be automatically determined, and determines that the first condition is satisfied (S102: YES) when the processing to be executed on the received medical image can be at least proposed or automatically determined.

As an example, it is determined that a medical image having a combination of a device for which the medical information processing device 1 has not yet determined processing and/or a parameter at the time of photographing for which the medical information processing device 1 has not yet determined processing does not satisfy the first condition.

Upon determining that the first condition is not satisfied (S102), the medical information processing device 1 proceeds to first processing (S104). The first processing is processing of determining processing input by a user as the processing to be executed on the received medical image, and storing the medical image and the determined processing in association with each other.

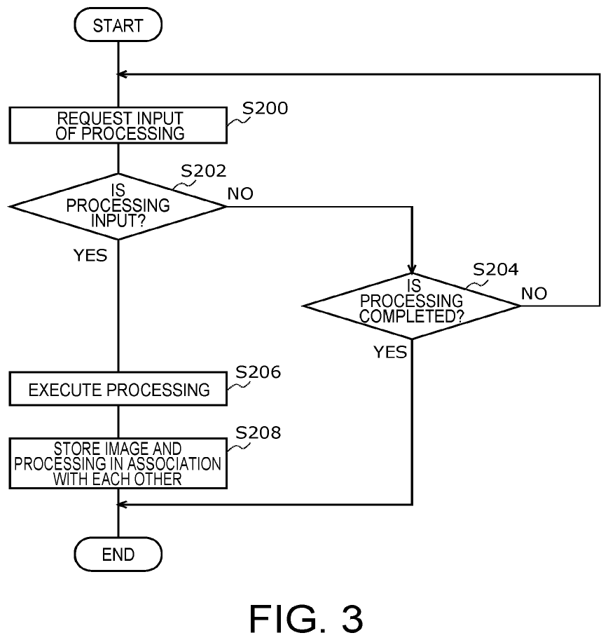

FIG. 3 is a flowchart illustrating an example of the first processing.

The processing input unit 106 requests the user to input processing (S200). The processing input unit 106 displays, for example, a received medical image itself, a parameter at the time of photographing read from the metadata, or the like, or both of the medical image and the parameter on the display unit 102, and causes the user to input processing to be executed via a user interface.

The processing input unit 106 determines whether the processing has been input (S202).

When the user does not input the processing for the received medical image to the processing input unit 106 (S202: NO), the processing input unit 106 may determine whether the processing has ended (S204).

Upon determining that the processing has ended (S204: YES), the medical information processing device 1 ends the processing. Upon determining that the processing has not ended (S204: NO), the medical information processing device 1 may proceed to the standby state of the processing of S200.

For example, when invalid processing has been received from the user or there has been no response from the user for a predetermined time, the processing input unit 106 may determine that the processing ends. The invalid processing includes, for example, an input of processing that cannot be executed in the medical information processing device 1 and an input of a parameter (including a combination) that cannot be applied in image processing.

When the processing input unit 106 receives processing applicable to the received medical image (S202: YES), the processing determination unit 112 determines that the processing input by the user is processing to be applied to the received medical image, and the processing execution unit 114 executes the processing determined by the processing determination unit 112 (S206).

The processing determination unit 112 stores the metadata and the determined (executed) processing in association with each other in the storage unit 104 (S208). As described above, in the first processing, an input from the user is received, processing is determined based on the input, and the determined processing is stored in the storage unit 104 as input processing data in association with the metadata of the received medical image.

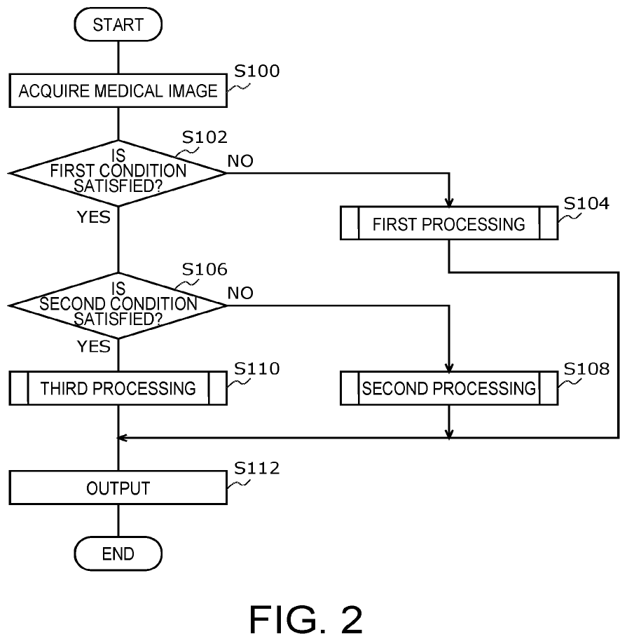

Referring back to FIG. 2, when the first condition is satisfied, that is, when the processing can be proposed for the received medical image and the processing cannot be automatically determined, the medical information processing device 1 determines whether a second condition is satisfied (S106). The second condition is a condition for determining, with respect to the received medical image, a state in which the processing for the received medical image can be automatically determined.

Upon determining that the first condition is satisfied and the second condition is not satisfied (S106: NO), the medical information processing device 1 proceeds to the second processing (S108). The second processing is processing of proposing processing to be executed from the medical information processing device 1, acquiring processing selected to be executed by the user, determining the acquired processing as processing to be applied to the medical image, and storing the medical image and the determined processing in association with each other.

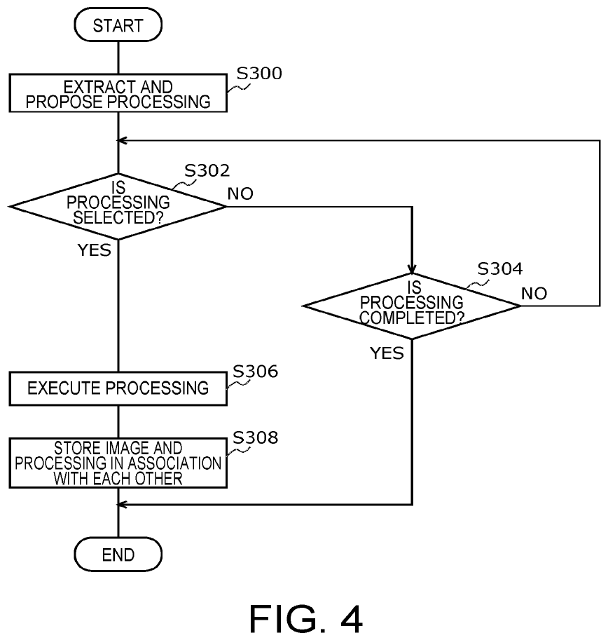

FIG. 4 is a flowchart illustrating an example of the second processing.

The processing proposal unit 108 extracts processing to be proposed from the metadata of the received medical image based on the data stored in the storage unit 104 and proposes the processing to the user (S300). The processing proposal unit 108 proposes the processing to the user, for example, by displaying the processing on the display unit 102.

The proposal result acquisition unit 110 acquires a result of processing selected by the user (S302).

As an example, the processing proposal unit 108 may propose one processing to be applied to the received medical image, and the proposal result acquisition unit 110 may acquire information indicating that the user has selected whether to apply the one processing.

As another example, the processing proposal unit 108 may propose a plurality of types of processing that may be applied to the received medical image, and the proposal result acquisition unit 110 may acquire one processing selected by the user from the plurality of types of processing.

When the processing is not selected (S302: NO), the proposal result acquisition unit 110 determines whether the processing has ended (S304), similarly to the processing in the first processing, and can wait for a result of the proposal of the processing or end the processing.

For example, when the user selects not to perform processing on one proposal or the user selects not to perform any processing on a plurality of proposals in the proposal result acquisition unit 110 (S302: NO), the processing determination unit 112 can also determine to end the proposal processing (S304: YES). In this case, the medical information processing device 1 may proceed to the first processing instead of the second processing.

When the processing is selected (S302: YES), the processing to be applied to the medical image received by the processing determination unit 112 is determined based on the processing acquired by the proposal result acquisition unit 110, and the processing execution unit 114 executes the processing (S306).

The processing determination unit 112 stores, in the storage unit 104, the metadata and the determined (executed) processing in association with each other (S308). As described above, in the second processing, the medical information processing device 1 proposes a proposal, receives user's selection for the proposal, determines processing based on the selection, and stores the determined processing in the storage unit 104 as proposal selection data in association with the metadata of the received medical image.

It is noted that the medical information processing device 1 may 1 may have an option of inputting another processing as a proposal selection in the second processing, and may add processing of executing the first processing when the user selects to input the processing.

Referring back to FIG. 2, when the second condition is satisfied, that is, when the processing to be executed on the received medical image can be automatically determined, the medical information processing device 1 executes third processing (S110). The third processing automatically determines processing to be executed on the medical image based on information related to the received medical image and data related to past processing stored in the storage unit 104, and executes the determined processing.

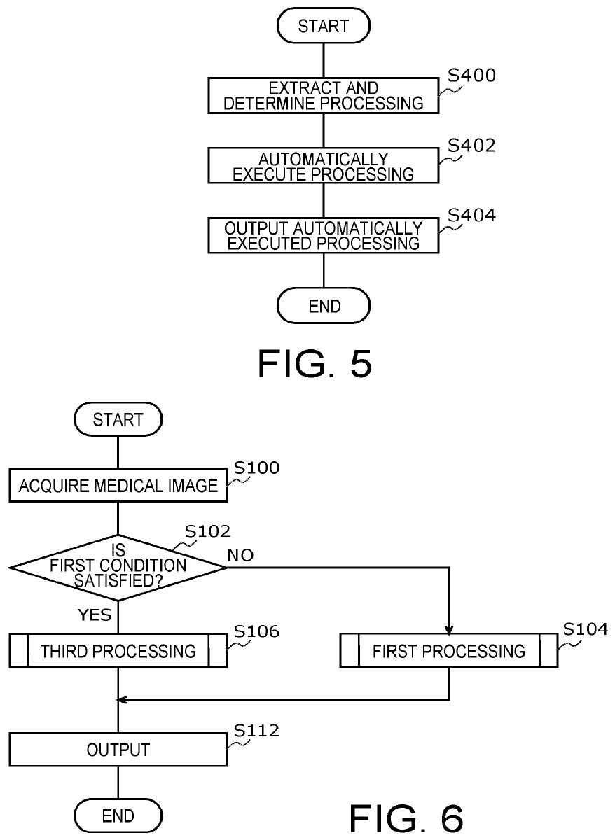

FIG. 5 is a flowchart illustrating an example of the third processing.

The processing determination unit 112 refers to the storage unit 104, extracts processing to be automatically executed associated with the data related to the received medical image, and determines processing to be applied to the medical image (S400). This processing is automatically executed without receiving an input and a selection from the user.

The processing execution unit 114 executes, on the received medical image, the processing determined by the processing determination unit 112 (S402).

When the processing is automatically executed on the received medical image, the processing execution unit 114 may output the automatically executed processing so that the user can recognize the processing (S404). For example, the processing execution unit 114 may display, on the display unit 102, the processing automatically executed on the medical image. The display may include, for example, information indicating that which rule is applied to execute the processing. In addition to an identifier of the rule and the like, this information can display which processing has been automatically executed by following a hyperlink and the like from the identifier.

In addition, the processing execution unit 114 may display at least an image after processing is automatically executed together with the automatically executed processing. In this case, the processing execution unit 114 can also display an image before the processing is automatically executed.

It is noted that the processing execution unit 114 can also output information indicating that the processing has been automatically executed by a printing output and a sound output or via any interface, instead of outputting the information indicating that the processing has been automatically executed by display.

Referring back to FIG. 2, the medical information processing device 1 outputs the medical image subjected to the first processing, the second processing, or the third processing from the output unit 116 (S112). As described above, the output may be a concept including outputting the medical image to the outside of the medical information processing device 1 or storing the medical image in the storage unit 104.

As described above, the medical information processing device 1 can execute, based on the information of the input medical image, the processing input by the user, proposed processing selected by the user, or the processing automatically determined.

Next, the first condition and the second condition will be described. The first condition is a condition for determining whether a user inputs processing to be executed on a medical image or the medical information processing device 1 proposes the processing. The second condition is a condition for determining whether to propose or automatically execute the processing to be executed on the medical image.

As a non-limiting example, the first condition may be a condition for determination based on whether there are a first predetermined number or more of information related to the past medical images and processing associated with the medical images. For example, when the same processing Pa has been input by the user for an image having a combination A of certain metadata a first predetermined number of times or more in the past, the medical information processing device 1 can perform control to propose this processing to the user in the processing proposal unit 108.

When a plurality of types of processing Pa1, Pa2, . . . are input for the combination A the first predetermined number of times or more, the processing proposal unit 108 can propose the plurality of types of processing to the user.

As another non-limiting example, the first condition may be a condition for determination based on whether information related to past medical images and processing associated with the medical images have been input to a second predetermined number or more of the medical information processing devices 1.

When a plurality of medical information processing devices 1 are provided as a part of the medical information processing system, it is also possible to use, as a determination condition, how many medical information processing devices 1 have received an input in this manner.

The same applies to the second condition, and the medical information processing device 1 can set, as the second condition, whether the processing has been selected a third predetermined number of times or more, or whether the processing has been selected in a fourth predetermined number or more of the medical information processing devices 1, based on association information between information of past medical images and processing proposed and executed for the medical images.

The first condition and/or the second condition may have flexibility with respect to the combination.

For example, the medical information processing device 1 can use, as a condition, processing for a medical image in which all pieces of metadata included in DICOM data in the received medical image match each other.

As another example, the medical information processing device 1 can set a condition in which a negligible parameter is set in the metadata included in the DICOM data in the received medical image.

As another example, the medical information processing device 1 can set a condition in which an allowable error range is set in each parameter of the metadata included in the DICOM data in the received medical image.

As another example, the medical information processing device 1 can input the metadata included in the DICOM data in the received medical image to a learned model so as to set a condition as to whether it is suitable for executing the same processing.

Although the DICOM data is referred to in the above description, data is not limited to the DICOM data, and any metadata may be used. In addition, the data may not be metadata, and may be data including information indicating a state of an image input together with the image.

As described above, with the medical information processing device 1 of the present embodiment, it is possible to go through the steps of (1) determining processing to be executed on a medical image by an input from a user and executing the processing, (2) proposing processing to the user & determining and executing the processing by selection of the user, and (3) automatically determining and executing processing.

Through such a processing step, it becomes possible to form a device that automatically determines processing that one or more users consider desirable for a medical image without performing programming in complex branches using DICOM data. This device can be formed by simple processing such as an input by a user with a keyboard or the like via a GUI or selection of a button or the like, and it is possible to reduce maintenance by an expert and reduce maintenance costs.

### Second Embodiment

In the above-described embodiment, the medical information processing device 1 proposes processing to a user, but may have a further simplified format.

FIG. 6 is a flowchart illustrating an example of processing of a medical information processing device 1 according to the embodiment. The processing denoted by the same reference numeral as that in FIG. 2 is basically the same processing as that in the first embodiment described above, and thus detailed description thereof will be omitted.

After acquiring a medical image (S100), the medical information processing device 1 determines whether the medical image satisfies a first condition (S120). The first condition is the same as that of the first embodiment as a condition, but whether to execute the first processing or the third processing as a determination result is different from that of the first embodiment.

That is, in the present embodiment, the first condition is a condition for determining a state in which processing to be executed on the received medical image can be automatically determined. In the present embodiment, the medical information processing device 1 determines, based on data acquired in the first processing in the past, whether to automatically determine the processing according to the first condition or to determine the processing according to an input from a user.

As described above, with the medical information processing device 1 of the present embodiment, it is possible to determine the processing to be automatically executed as two-step processing.

It is noted that, in the above description, the number of processing steps is reduced as compared with that of the first embodiment, but the present invention is not limited thereto. The medical information processing device 1 according to the present embodiment can increase the step of processing as another example.

For example, the medical information processing device 1 can further increase at least one of the first processing and the second processing described in the first embodiment in two or more steps.

For example, the medical information processing device 1 executes processing similar to the first processing described above as a first step of the first processing. Thereafter, the medical information processing device 1 may include a second step of the first processing of selecting processing based on data acquired in the first step of the first processing in the past and receiving an input of a parameter or the like in the selected processing from the user.

As the determination of a branch between the first step and the second step of the first processing, for example, the number of times the processing input from the user is equivalent processing or the number of medical information processing devices 1 to which the processing is input from the user among the plurality of medical information processing devices 1 can be used.

For example, the medical information processing device 1 proposes a plurality of types of processing in the above-described second processing to the user as the first step of the second processing, and selects and determines processing to be executed by the user from the plurality of types of processing. Thereafter, the medical information processing device 1 may propose one processing to the user based on the processing associated in the first step of the second processing in the past, and cause the user to select whether to determine the processing as the processing to be executed.

As the determination of a branch between the first step and the second step of the second processing, for example, the number of times the user has selected a proposal among the plurality of proposals or the selected number of the medical information processing devices 1 can be used. Furthermore, in the first step of the second processing, three or more types of processing may be proposed, and one or a plurality of steps of gradually reducing the proposed processing may be provided between the first step and the second step of the second processing based on the selected number of times or the number of devices.

### Third Embodiment

A medical information processing device 1 may execute determined processing on a medical image by calling, for each processing, an application for executing the processing.

As another example, the application may be provided in another medical information processing device, and in this case, the medical information processing device 1 may transmit an image and information on the determined processing to the other medical information processing device, and execute the processing in the other medical information processing device.

Three or more medical information processing devices may be provided.

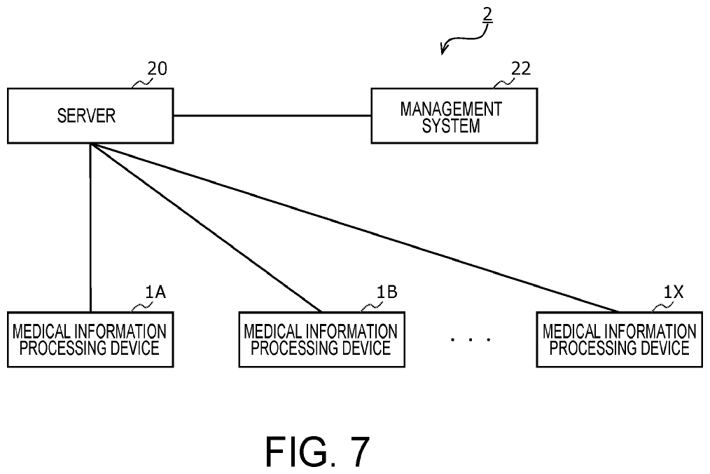

FIG. 7 is a block diagram schematically illustrating an example of a medical information processing system including a plurality of medical information processing devices 1 according to the embodiment. A medical information processing system 2 includes a plurality of medical information processing device 1A, 1B, . . . , and 1X, a server 20, and a management system 22.

Each medical information processing system has a function equivalent to that of the medical information processing system described above.

The server 20 is connected to each of the medical information processing devices and controls these medical information processing devices. In this embodiment, among the storage units 104 described in the above-described embodiments, the storage unit that stores past data is provided in the server 20 and can comprehensively manage association between processing determined in each medical information processing device and information related to a medical image.

That is, the medical information processing device can include a transmission unit that transmits, to the outside, the medical image subjected to processing determined by an input, determined by a proposed result selection, or automatically determined. This transmission unit can function as a transmission unit in the medical information processing device 1.

It is noted that the server 20 is not limited to being provided independently, and may be provided in a form in which one or a plurality of medical information processing devices among the plurality of medical information processing devices 1 can also operate as a server.

The management system 22 is a system that manages medical information connected to the server 20. The management system 22 may be, for example, a system such as a picture archiving and communication system (PACS), and the server 20 can manage the processed medical image in the management system 22.

It is noted that, although the server 20 and the management system 22 are connected to each other in the drawing, the present invention is not limited to this form, and an individual medical information processing device may directly transmit data to the management system 22.

As described above, with the medical information processing system of the present embodiment, it is possible to construct a system that determines more comprehensive automatic processing. This medical information processing system can be constructed using, for example, a plurality of medical information processing devices in a limited environment such as in a hospital, or conditions constructed in one medical information processing system 2 can be shared with other medical information processing systems, thereby making it possible to form a more global control. In this case, when information attached to the medical image includes patient information, it is possible to share the information while protecting privacy, and when the information includes information related to an individual difference of the imaging device, it is also possible to share a condition in which a condition regarding the individual difference is mitigated.

In the above-described embodiment, the input interface can be implemented by a trackball for performing various settings, a switch button, a mouse, a keyboard, a touch pad for performing an input operation by touching an operation surface, a touch screen in which a display screen and the touch pad are formed to be integrated with each other, a non-contact input circuit using an optical sensor, a sound input circuit, and the like. The input interface is connected to a control circuit, converts an input operation received from an operator into an electrical signal, and outputs the electrical signal to the control circuit. It is noted that, in the present specification, the input interface is not limited to one including physical operation components such as a mouse and a keyboard. For example, examples of the input interface include an electrical signal processing circuit that receives an electrical signal corresponding to an input operation from an external input device provided separately from the device and outputs the electrical signal to the control circuit.

In the above-described embodiment, each processing function of the information processing function is recorded in a storage circuit in the form of a program executable by a computer. The processing circuit may include a processor. For example, the processing circuit reads the program from the storage circuit and executes the program to implement a function corresponding to each program. In other words, the processing circuit in a state of reading each program has each function illustrated in the processing circuit illustrated in the drawings. It is noted that, in the drawings, it has been described that each processing function is implemented by a single processor. However, the processing circuit may be configured by combining a plurality of independent processors, and functions thereof may be implemented by allowing each processor to execute the program. Furthermore, in the drawings, it has been described that a single storage circuit stores a program corresponding to each processing function, but a plurality of storage circuits may be arranged in a distributed manner, and the processing circuit may read the corresponding program from each storage circuit.

In the above description, an example has been described in which the “processor” reads the program corresponding to each function from the storage circuit and executes the program, but the embodiment is not limited thereto. The term “processor” can mean, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). In a case where the processor is, for example, the CPU, the processor implements a function by reading and executing the program stored in the storage circuit. On the other hand, when the processor is the ASIC, instead of storing the program in the storage circuit, the function is directly incorporated as a logic circuit in a circuit of the processor. It is noted that each processor of the present embodiment is not limited to being configured as a single circuit for each processor, and a plurality of independent circuits may be combined to be configured as one processor to implement the function. Further, a plurality of components in the drawings may be integrated into one processor to implement the function.

According to at least one embodiment described above, image processing of a medical image can be appropriately executed automatically or semi-automatically.

Although several embodiments have been described, these embodiments have been presented as examples, and are not intended to limit the scope of the invention. These embodiments can be implemented in various other forms, and various omissions, substitutions, changes, and combinations of the embodiments can be made without departing from the gist of the invention. These embodiments and modifications thereof are included in the scope and gist of the invention and are included in the invention described in the claims and the scope equivalent thereto.

1. A medical information processing device comprising:
a memory; and
a processor configured to:
receive a medical image;
store, in the memory, information on the medical image and information on processing executed on the medical image in association with each other;
determine processing to be executed based on the information on the medical image and the information on the processing stored in the memory; and
execute, upon receiving the medical image, the processing to be executed determined based on the information on the medical image.

2. The medical information processing device according to claim 1, wherein
the processor is configured to perform,
as first processing,
determining processing input by a user as the processing to be executed on the medical image, and
storing, in the memory, information on the determined processing for the medical image.

3. The medical information processing device according to claim 2, wherein
the processor is further configured to:
propose, to the user, one or a plurality of types of processing for the medical image based on the processing executed by an input of the user and stored in the memory;
acquire information on the processing selected by the user among the proposed processing; and
when the information on the processing acquired in the first processing in the past satisfies a first condition with respect to the information on the input medical image,
perform, as second processing,
determining the acquired processing selected by the user as the processing to be executed on the input medical image, and
storing, in the memory, the acquired processing selected by the user in association with the information on the input medical image.

4. The medical information processing device according to claim 3, wherein
the processor is further configured to,
when information on the processing acquired in the second processing in the past satisfies a second condition with respect to the information on the input medical image,
perform, as the second processing,
determining processing to be automatically executed on the input medical image based on the information on the input medical image and data related to past processing stored in the memory.

5. The medical information processing device according to claim 2, wherein
the processor is further configured to,
when the information on the processing acquired in the first processing in the past satisfies a first condition with respect to the information on the input medical image,
perform, as third processing,
determining processing to be automatically executed on the input medical image based on the information on the input medical image and data related to past processing stored in the memory.

6. The medical information processing device according to claim 1, wherein:
the medical image is data including metadata; and
the information on the medical image is information extracted from the metadata.

7. The medical information processing device according to claim 1, further comprising
a transmission unit configured to transmit the processed medical image to an outside.

8. The medical information processing device according to claim 1, wherein:
the memory stores the processing executed on the medical image in a plurality of the medical information processing devices; and
the processor is configured to
select, as processing to be applied to the medical image, the processing executed on the medical image in a predetermined number or more of the medical information processing devices.

9. The medical information processing device according to claim 1, wherein
the processor is configured to
select, as processing to be applied to the medical image, the processing executed on the medical image a predetermined number of times or more.

10. A medical information processing system comprising:
one or a plurality of medical information processing devices; and
one or a plurality of storage devices, wherein:
the storage device stores information on processing executed on a medical image in the medical information processing device; and
the medical information processing device proposes processing to be executed on the medical image to a user or automatically determines the processing to be executed on the medical image based on the processing executed on the medical image and stored in the storage device.

11. A non-transitory computer readable medium having a program stored therein and configured to cause a computer to execute:
receiving a medical image;
storing, in a memory, processing executed on the medical image;
determining processing to be executed based on the medical image and data stored in the memory; and
automatically executing, upon receiving the medical image, the determined processing.
